(19) United States
(12) Patent Application Publication
(10) Pub. No.: US 2018/0059107 A1
(43) Pub. Date: Mar. 1, 2018

(54) METHOD OF DETECTING OR DIAGNOSING SJOGREN'S SYNDROME BASED ON THE PRESENCE OF AUTO-ANTIBODY AGAINST AQUAPORIN 5

(71) Applicant: Seoul National University R&DB Foundation, Seoul (KR)

(72) Inventors: Youngnim CHOI, Seoul (KR); Jahan ALAM, Seoul (KR)

(73) Assignee: Seoul National University R&DB Foundation, Seoul (KR)

(21) Appl. No.: 15/796,059

(22) Filed: Oct. 27, 2017

Related U.S. Application Data
(63) Continuation-in-part of application No. PCT/KR2015/011741, filed on Nov. 4, 2015.

(30) Foreign Application Priority Data
Apr. 29, 2015 (KR) 10-2015-0060344

(51) Int. Cl. G01N 33/564

(52) U.S. Cl. CPC G01N 33/564; G01N 2333/705; G01N 2800/24

(57) ABSTRACT

Disclosed are methods, compositions and kits of detecting or diagnosing Sjogren's syndrome based on the presence of autoantibody against aquaporin 5, which is found in the present disclosure. The present methods, compositions or kits can be advantageously used for accurate, reliable and rapid diagnose of Sjogren's syndrome, which is known to be difficult by conventional methods.

### CROSS-REFERENCE TO RELATED APPLICATIONS

The present application is a continuation in part application of International Patent Application No. PCT/KR2015/011741, filed Nov. 4, 2015, and claims the benefit of Korean Patent Application No. 2015-0060344, filed Apr. 29, 2015 in the Korean Intellectual Property Office, the disclosure of which are incorporated herein.

### STATEMENT OF GOVERNMENT SUPPORT

The invention was made with government support under grant number HI13C0016, “the Korean Health Technology R&D Project” awarded by the Ministry of Health & Welfare, Republic of Korea.

### BACKGROUND

### Field of the Invention

The present application is a technology related to the diagnosis of Sjogren's syndrome based on the autoantibody found in patient with Sjogren's syndrome.

### Description of the Related Art

Sjogren's syndrome is a disease characterized mainly by dryness of the mouth and eyes and by causing a reduction in the function of salivary and lacrimal glands by autoimmune responses. According to 2016 American College of Rheumatology/European League Against Rheumatism criteria for diagnosis of Sjogren's syndrome include: 1) focal lymphocyte sialadenitis in labial salivary gland with focus score≧1; 2) positivity of anti-SSA/Ro autoantibodies; 3) ocular staining score≧5; 4) Schirmer test≦5 mm/5 minutes in at least 1 eye; and 5) unstimulated whole saliva flow rate≦0.1 ml/minute.

U.S. Pat. No. 8,765,387 relates to a method for diagnosing Sjogren's syndrome and discloses a method of diagnosing Sjogren's syndrome by testing antibodies against SP-I, PSP and CA6.

However, there does not exist diagnostic markers that can explain the cause for a reduction in the function of salivary and lacrimal glands among the diagnostic criteria currently used and that can be related to the disease activity. Thus there is a need for the development of biological markers for diagnosis with such properties.

### SUMMARY OF THE INVENTION

The present disclosure is intended to demonstrate the etiology of Sjogren's syndrome and to provide a marker reflecting the activity of the disease and the use thereof.

In one aspect, the present disclosure provides a method of detecting an anti-aquaporin 5 auto-antibody present in a sample in order to diagnose Sjogren's syndrome.

In one embodiment, the method is performed based on the detection of Sjogren's syndrome-specific autoantibody found in the present disclosure, and comprises the steps of providing an aquaporin 5 antigen; contacting the sample in need of the detection with the antigen, and detecting whether the auto-antibody is present in the sample by detecting the binding between the antigen and the auto-antibody.

In one embodiment the presence of the binding between the antigen and the antibody in comparison to the result of the negative control indicates the presence of Sjogren's syndrome in the patient from which the sample is obtained.

In another aspect, the present disclosure also provides a kit for diagnosing or detecting Sjogren's syndrome, the kit comprising an aquaporin 5 antigen and a detection antibody, wherein the detection antibody binds specifically to human IgG or IgA, and is labeled with a chromophore; an enzyme comprising alkaline phosphatase, biotin, beta-galactosidase or peroxidase; a radioisotope; or a material comprising colored particles, including colloidal gold particles or colored latex particles.

In another aspect, the present disclosure also provides a composition for detecting Sjogren's syndrome, comprising an aquaporin 5 protein or its fragment or a cell that expresses the protein.

The method, kit or composition according to the present disclosure comprises antigen-antibody complex detection which may be performed by, for example, FACS (fluorescence-activated cell sorting), cellular immunostaining, radial immunodiffusion, an immunoprecipitation assay including immunoelectrophoresis or countercurrent electrophoresis, RIA (radioimmunoassay) or ELISA (enzyme linked immunosorbent assay).

In one embodiment, the antigen-antibody binding detection in the present disclosure involves the use of a detection antibody, wherein the detection antibody binds specifically to human IgG or IgA, and the detection antibody is labeled with a chromophore; an enzyme comprising alkaline phosphatase, biotin, beta-galactosidase or peroxidase; a radioisotope; or a material comprising colored particles, including colloidal gold particles or colored latex particles.

The antigen in the method, composition or kit according to the present disclosure may be provided in the form of full-length aquaporin 5 proteins as a transmembrane protein present in a cell which does not express an endogenous aquaporin 5 or as a soluble form including extracellular loops of the aquaporin 5.

The sample that is used in the present disclosure may be a whole blood, a serum, a plasma, a tear, or a saliva.

The present disclosure discloses a method or kit of detecting or diagnosing Sjogren's syndrome based on the detecting the presence of aquaporin 5 auto-antibody specifically found in SS patients. The method or kit according to the present disclosure can accurately and rapidly diagnose Sjogren's syndrome whose diagnosis is difficult by conventional methods, and can also play an important role in evaluating post-treatment therapeutic effects by demonstrating the etiology of the disease and reflecting the activity of the disease.

In other aspect, the present disclosure provides a method of diagnosing Sjogren's syndrome in a subject in need thereof, the method comprising: obtaining a biological sample from the subject; and contacting the sample with an aquaporin 5 antigen to allow a complex formation between the aquaporin 5 and auto-antibodies to aquaporin 5 which may be present in the sample; detecting the complex; and diagnosing the patient having SS when the complex is detected.

### BRIEF DESCRIPTION OF THE FIGURES

FIG. 1A shows higher levels of anti-AQP5 IgG were detected in the SS sera by indirect immunofluorescence assay in which Madin-Darby Canine Kidney Epithelial (MDCK) cells over-expressing AQP5 were stained with anti-AQP5 antibodies and various dilutions of either the control or SS sera, followed by Alexa Fluor 488-conjugated anti-goat IgG(green) and CFTM 594-conjugated anti-human IgG (red). As controls, healthy human serum was used.

FIG. 1B shows the graph in which the intensities of the red signals for anti-AQP5 IgG were expressed by the magnitude of brightness that was reduced until the staining of AQP5 disappeared (left panel) and a receiver operating characteristic (ROC) curve for the levels of anti-AQP5 IgG (right panel). The arrow indicates the value used for cutoff 2 in the left panel.

### DETAILED DESCRIPTION OF THE EMBODIMENTS

The present disclosure is based on the finding that aquaporin 5 auto-antibody is produced specifically in Sjogren's syndrome patients and may be used as a major diagnostic marker that reflects the activity of the disease.

Therefore, in one aspect, the present disclosure is directed to a method of detecting an anti-aquaporin 5 auto-antibody based on the presence of Sjogren's syndrome patient specific auto-antibody in order to diagnose Sjogren's syndrome, the method comprising the steps of: providing an aquaporin 5 antigen; bringing the antigen into contact with a sample or a biological sample, in which an antigen-antibody complex is formed by the contact; and detecting whether the auto-antibody is present by detecting the antigen-antibody complex or the binding therebetween.

In another aspect, the present disclosure is directed to a composition for diagnosing Sjogren's syndrome, comprising aquaporin 5.

The method or composition according to the present disclosure can be used for diagnosis of onset of Sjogren's syndrome, diagnosis of progression of the disease, or determination of responses to therapeutics. In one embodiment, the detection of the presence of the autoantibody to aquaporin 5 indicates that the subject from which the sample is obtained is having or diagnosed as Sjogren's syndrome.

Sjogren's syndrome (SS) is a disease characterized mainly by dryness of the mouth and eyes, and causes a reduction in function of salivary and lacrimal glands by autoimmune responses. Aquaporin 5 is a water-specific channel protein that is expressed in the acinar cells and duct cells of the salivary and lacrimal glands, and provides a channel through which water molecules pass through the cell membrane consisting of lipids.

The method, composition or kit according to the present disclosure can be advantageously used for diagnosis of Sjogren's syndrome. As used herein, the term “diagnosis” is meant to include determining the susceptibility of a subject to a specific disease or disorder, or determining whether a subject currently has a specific disease or disorder, or determining the prognosis of a subject suffering from a specific disease or disorder (for example, determining the progression stage of the disease or determining the responsiveness of the disease to treatment), or therametrics (for example, monitoring the status of a subject in order to provide information about therapeutic efficacy).

As used herein, the term “detection” is meant to include quantitative/qualitative analysis that includes the detection of presence or absence, or the concentration measurement. Such methods for detecting are known in the art, and one of ordinary person in the art would be able to select appropriate methods to practice the present disclosure.

The methods, compositions or kits according to the present disclosure are based on the findings that auto-antibody against aquaporin 5 are present in a subject with SS disease. Thus as long as aquaporin 5 that functions as an antigen to detect the auto-antibody, various aquaporin 5 can be used to detect the auto-antibody. For example, aquaporin 5 known in the art may be used, which can be synthesized, isolated and purified by conventional methods. For example, the antigen includes a sequence from mammals, particularly humans, for example, with GenBank Accession No. NM_001651.3 (mRNA), NP_001642.1 (protein), but is not limited thereto. The full-length protein or a fragment thereof, particular extracellular domain/region/loop may be used, which can be found easily at the level of ordinary skill in the art from the public sequence database. Also included is a functional equivalent thereof.

In an embodiment of the present disclosure, as a transmembrane protein, the aquaporin 5 antigen may be provided in the form of cells expressing aquaporin 5 in the membrane, particularly as a transmembrane protein. In one embodiment, for such cells, cells that do not express any innate aquaporins, including aquaporin 5, are particularly used. For the expression of aquaporin 5 proteins in cells, a plasmid expressing aquaporin 5 is transfected into the corresponding cells and expressed in the cells in which the aquaporin 5 proteins are present as a transmembrane protein. For example, MDCK cells as described in the Examples of the present disclosure may be used, but are not limited thereto. When the aquaporin 5 antigen is provided in the form of cells expressing aquaporin 5 in the membrane, the choice of proper fixative and a method for antigen retrieval is important for successful detection of auto-antibody to aquaporin 5. The fixation with alcohol may reduce the amount of aquaporin 5 in the membrane. Also the fixation with formalin or paraformaldehyde requires antigen retrieval. In the present disclosure, it is found that fixation with 4% paraformaldehyde with heat-induced antigen retrieval is an optimal condition that differentiates patient samples from control ones.

In other embodiments, when aquaporin 5 may be used in a soluble form, the extracellular loops of aquaporin 5 are used. When the soluble form of aquaporin 5 is used as antigen, the skill to keep the right conformation is important for successful detection of auto-antibody to aquaporin 5.

The sample or biological sample according to the present disclosure is a sample from a person suspected to have Sjogren's syndrome, or a person suffering from Sjogren's syndrome, or a person which has received treatment after diagnosed to have Sjogren's syndrome.

This sample is not particularly limited, as long as anti-aquaporin 5 auto-antibody is detected. The term “sample” refers to a material or a mixture of materials, which contains one or more detectable components. Examples of the sample include, but are not limited to, cells, tissues or body fluids from organisms, particularly humans, for example, body fluids such as salvia or tears, whole blood, plasma, and serum. In one embodiment of the present disclosure, blood, particularly serum, is used.

In the method according to the present disclosure, the antigen-antibody complex may be detected using various known methods. For example, the antigen-antibody complex may be detected by radial immunodiffusion, an immunoprecipitation assay including immunoelectrophoresis or countercurrent electrophoresis, RIA (radioimmunoassay), or ELISA (enzyme linked immunosorbent assay). This detection method is particularly advantageous when the antigen is provided as a soluble protein. In one embodiment, the extracellular protein domain of aquaporin 5 may be provided as the antigen. In one embodiment of the present disclosure, an ELISA method, particularly sandwich-type ELISA, is used, and in this case, a detection antibody as described below is also used.

In other embodiments, when the antigen is provided in the form of cells, cell-based assay methods may be used, for example, FACS (fluorescence-activated cell sorting) assay, or cellular immunostaining with antibody, for example, immunofluorescence cell staining as described in examples of the present disclosure.

Through the process of detecting the antigen-antibody complex, the intensity of the final signal of the sample is analyzed and compared with that of the signal of a normal control sample. When the signal intensity is higher than that of the normal control sample, the sample may be diagnosed as Sjogren's syndrome, or treatment progress may be determined.

In one embodiment of the present disclosure, for detection of the antigen-antibody complex, the antigen is labeled with a label as described below, or a detection antibody or secondary antibody is used.

The detection antibody that may be used in the method according to the present disclosure binds specifically to human IgG or IgA. The detection antibody may be labeled with a substance that can be detected visually or using various image detection systems.

In one embodiment of the present disclosure, the antigen or detection antibody according to the present disclosure may be labeled with an enzyme that can catalyze a chemical reaction in the presence of a specific substrate to produce a color reaction or emit light. Examples of the enzyme include, but are not limited to, peroxidases such as horseradish peroxidase, alkaline phosphatase, glucose oxidase, beta-galactosidase, urease, catalase, asparginase, ribonuclease, malate dehydrogenase, staphylococcal nuclease, triosephosphate isomerase, glucose-6-phosphate dehydrogenase, glucoamylase, and acetylcholine esterase.

In other embodiments, the antigen or detection antibody according to the present disclosure may be labeled with a chromophore that emits light having a wavelength different from that of irradiated light by light irradiation and that is used in bioluminescence, chemiluminescence, electroluminescence, electrochemiluminescence, and photoluminescence. Examples of the chromophore include, but are not limited to, proteins, including green fluorescence protein; and organic compounds, including fluorescein isothiocyanate, rhodamine, phycoerythrin, phycocyanin, allophycocyanin, and fluorecamine.

In another embodiment, the antigen or detection antibody according to the present disclosure may be labeled with various radioisotopes.

In the present disclosure, when the label is, for example, a radioisotope, detection of the label may be performed by a scintillation counter, and when the label is a fluorescent substance, detection of the label may be performed by a method such as spectroscopy, a phosphoimaging system or a fluorescence counter. When the label is an enzyme, detection of the label may be performed by measuring a chromogenic product that occurs by enzymatic conversion of a suitable chromogenic substrate. Furthermore, detection may be performed by comparing the color of a chromogenic product that occurs by an enzymatic reaction, through comparison with a suitable standard or control.

In one embodiment, the antigen or detection antibody according to the present disclosure comprises, for example, a chromophore; an enzyme including alkaline phosphatase, biotin, beta-galactosidase or peroxidase; a radioisotope; or a material comprising nanoparticles such as colloidal gold particles or colored latex particles, but is not limited thereto.

In another aspect, the present disclosure also provides a kit for detecting or diagnosing Sjogren's syndrome, which is used in the method according to the present disclosure and comprises a solid support having aquaporin 5 antigen adsorbed thereon and a detection antibody, in which the detection antibody binds specifically to human IgG, and the antigen or detection antibody is labeled with a chromophore; an enzyme including alkaline phosphatase, biotin, beta-galactosidase or peroxidase; a radioisotope; or a material comprising nanoparticles, including colloidal gold particles or colored latex particles.

For one that overlaps the above-described components, among the components that are included in the kit according to the present disclosure, reference may be made to the foregoing description.

In the kit according to the present disclosure, the antigen according to the present disclosure may be attached to a microwell plate such as a 96-well microwell plate, beads or particles, including colloidal gold particles or colored latex particles, or a membrane such as a cellulose, nitrocellulose, polyethersulfone, polyvinylidene, fluoride, nylon, charged nylon or polytetrafluoroethylene membrane. For attachment or coating of the antigen, a known method may be used. For example, attachment or coating of the antigen may be performed with reference to the method descried in examples of the present disclosure.

In one embodiment, the method or kit according to the present disclosure may be used in a sandwich-type immunoassay such as ELISA (Enzyme Linked Immuno Sorbent assay), RIA (Radio Immuno Assay) or the like. In this method, a sample may be added to an antigen bound to a solid support, for example, a bead, membrane, slide or microwell plate made of glass, plastic (e.g., polystyrene), polysaccharide, nylon or nitrocellulose, and then an antigen-antibody complex may be qualitatively or quantitatively detected by antigen binding to an antibody either labeled with a directly or indirectly detectable label, for example, a radioisotope such as ³H or ¹²⁵I as described above, a fluorescent substance, a chemiluminescent substance, heptene, biotin, digoxigenin or the like, or conjugated with an enzyme such as horseradish peroxidase, alkaline phosphatase or malate dehydrogenase, which can develop color or emit light by its reaction with a substrate. Furthermore, the immunoassay method is described in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, N J, 1984, etc. The ELISA kit may further comprise reagents capable of detecting the bound antibody, for example, a secondary detection antibody labeled with the above-described substance such as a chromophore, an enzyme (e.g., conjugated with antibody) or the like, and a substrate that is used in detection.

In other embodiments, the method or kit according to the present disclosure may be used in the form of arrays, including microarrays, or chips. The antigen may be attached to the surface of a support such as glass or nitrocellulose. For array fabrication technology, reference may be made to, for example, Schena et al., 1996, Proc Natl Acad Sci USA. 93(20):10614-9; Schena et al., 1995, Science 270(5235):467-70; and U.S. Pat. Nos. 5,599,695, 5,556,752 or 5,631,734. As a fluorescence spectrometer, a scanning confocal microscope may be used and is available from, for example, Affymetrix, Inc. or Agilent Technologies, Inc.

In addition, the method or kit according to the present disclosure is used in the form of a dip stick rapid kit depending on the pattern of analysis. The dip stick rapid kit is a technology that is widely used in the field of POCT (Point of Care Treatment). In the case of the dip stick rapid kit, the antigen according to the present disclosure is bound to a support such as nitrocellulose, and when the antigen is brought into contact with a sample (for example, one end of the dip stick is dipped in a serum sample), the sample moves a substrate by a capillary phenomenon so that the antigen will bind to an antibody in the substrate to develop color. In this manner, the antigen-antibody complex is detected.

Furthermore, the kit according to the present disclosure may be used in a lateral flow assay depending on the pattern of analysis. The lateral flow assay is a method for quantitatively or qualitatively measuring a specific substance (e.g., a specific nucleic acid or protein) contained in a sample. For example, in the lateral flow assay, using a nitrocellulose membrane (medium for development) having an antigen bound at a specific position, the substance to be analyzed is moved by a chromatographic method, and a specific nucleic acid or protein in the sample is detected by an antigen-antibody reaction.

Furthermore, the kit according to the present disclosure may comprise a guidebook about a method of using the biomarker according to the present disclosure. In addition, the kit may further comprise a reagent or the like, which can detect an antibody specific for a control, or a bound antibody.

Hereinafter, examples will be presented to help understand the present disclosure. It is to be understood, however, that these examples are provided to merely facilitate the understanding of the present disclosure and are not intended to limit the scope of the present disclosure.

### EXAMPLES

### Example 1: Preparation of MDCK Cell Line (MDCK-AQP5) Overexpressing Aquaporin 5 (AQP5)

cDNA (GenBank No: NM_001651) encoding AQP5 was obtained from professor Kyung-Pyo Park, Seoul National University School of Dentistry, and cloned into a pcDNA3.1 vector (Invitrogen, USA) to obtain a pcDNA3.1-AQP5 plasmid.

Next, the plasmid was transfected into MDCK cells (Madin-Darby Canine Kidney Epithelial Cells, Korean Cell Line Bank) by a calcium phosphate method. Then, the cells were cultured in MEM (Minimum Essential Medium) containing G418 (Sigma) under the conditions of 5% CO₂ and 37° C., and cells specifically expressing aquaporin 5 were selected.

### Example 2: Collection of Patient Serum Samples

This study was performed in compliance with the Helsinki Declaration after obtaining approvals from the Institutional Review Board of Seoul National University Hospital, the Institutional Review Board of Seoul National University Dental Hospital and the Institutional Review Board of Seoul St. Mary's Hospital. Sera were obtained from two patient groups: 1) 10 primary SS patients registered in the Seoul National University Rheumatism Clinic, who were diagnosed as primary SS patients according to the 2002 American European Consensus Group (AECG) classification criteria; and 2) 102 enrolled at the Korean Initiative of primary Sjögren's Syndrome (KISS) who fulfilled the 2002 American-European Consensus group (AECG) classification criteria and/or the 2012 American College of Rheumatology (ACR) criteria. Sera were collected before the start of treatment. Furthermore, resting and stimulated whole salivary flow rates were measured by spiting and masticatory stimuli using wax gum (GC America Inc. USA) (Navazesh M et al., J Dent Res. 1982; 61:1158-62). All patients were females with ages ranging from 21 to 80 years (mean age 52.5±10.7 years). Control sera were obtained from healthy females who did not show any signs or symptoms of SS. Subjects with systemic diseases other than hypertension were excluded. Clinical data and samples were obtained after obtaining written informed consent from all patients.

### Example 3: Immunofluorescent Staining for Detection of Marker According to the Present Disclosure

The MDCK-AQP5 cells prepared in Example 1 were incubated on a coverslip. Then, the MDCK-AQP5 cells were stimulated with 400 μM cAMP (Sigma) for 24 hours to induce translocation of AQP5 from the cytoplasm to the cell membrane.

Then, the incubated cells were fixed with 4% paraformaldehyde for 0.5 hours. For antigen retrieval in the fixed cells, sodium citrate buffer (10 mM sodium citrate, 0.05% Tween-20, pH 6.0) was added to the cells, followed by heating at 105° C. for 20 minutes.

After blocking cells with 5% BSA, primary antibody was added to the cells, and the cells were incubated overnight at 4° C. As the primary antibody, a 1:100 dilution of goat AQP5-specific antibody (Santa Cruz, USA) and a 1:200 dilution of patient or normal control serum were used. A total of 53 control sera and 112 patient sera were analyzed. Then, the primary antibody and unbound antibody in the incubated cells were washed three times with PBS. Then, as secondary antibodies, Alexa Fluor 488-conjugated donkey anti-goat IgG antibody (Santa Cruz) and CF-594-conjugated anti-human IgG antibody (Sigma) were added to the cells, and the cells were incubated at room temperature for 2 hours, after which unbound secondary antibodies were washed three times with distilled water. The cells were examined by confocal microscopy (Carl Zeiss, 400×).

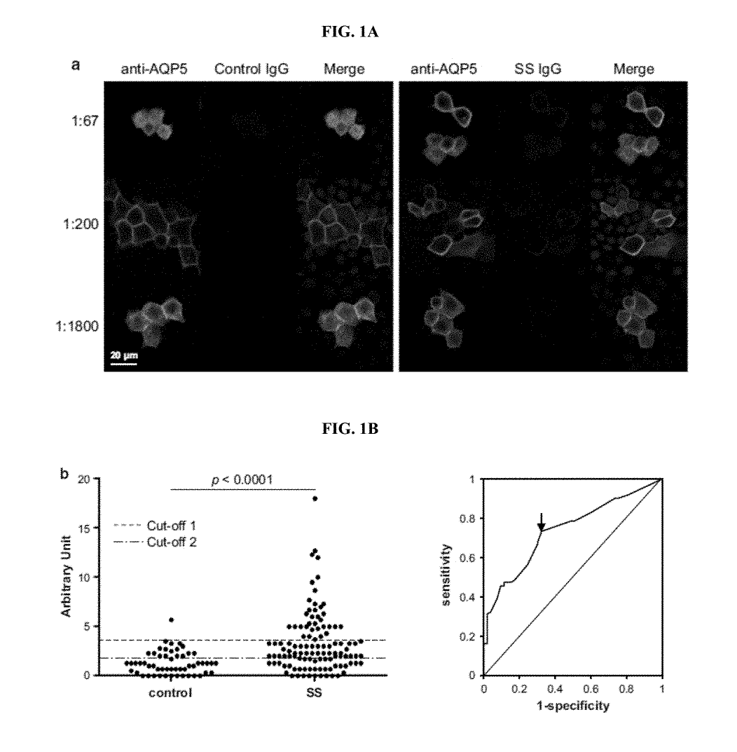

The results are shown in FIG. 1. As shown therein, the results of AQP5-specific antibody staining indicated that AQP5 was located mainly in the cell membrane and was partially expressed in the cytoplasm. Furthermore, it can be seen that control serum did not stain the MDCK-AQP5 cells, whereas the serum containing anti-AQP5 auto-antibody from the patient with Sjogren's syndrome stained the MDCK cells expressing AQP5, and the signal overlapped with that of the AQP5-specific antibody.

Further analyses of 53 control sera and 112 patient sera in two different reference point settings, following results as shown in Table 1 were obtained. Table 1 shows the detection frequency of anti-aquaporin 5 auto-antibody in controls and Sjogren's syndrome patients, and the detection frequencies are expressed as the number of cases detected (%).

TABLE 1

| | Control (n = 53) | | Sjogren's syndrome (n = 112) | |
|---|---|---|---|---|
| Reference point 1 | 1 | (1.9%) | 35 | (31.3%) |
| Reference point 2 | 17 | (32.1%) | 82 | (73.2%) |

### Example 4: Determination of the Relationship of Anti-AQP5 Auto-Antibody Marker with Sjogren's Syndrome in Comparison with Conventional Anti-Ro and Anti-La

### IgG Markers

The auto-antibody to AQP5 marker according to the present disclosure was compared with anti-Ro and anti-La IgG, which are conventionally used markers for diagnosis of Sjogren's syndrome, by analyzing the relationship with the salivary flow in patients.

Anti-AQP5 detection was performed as described in Example 3. Anti-Ro and anti-La IgG were determined as the part of diagnostic tests in hospitals. The results are shown in Table 2 below. As shown therein, resting salivary flow in patients with anti-AQ5 IgG or IgA was significantly lower than those from patients without anti-AQ5 auto-antibody. However, it is shown that anti-Ro and anti-La IgG, conventionally used markers for diagnosis of Sjogren's syndrome, were not related to the salivary flow. Such results indicates that the anti-AQP5 auto-antibody according to the present disclosure is highly likely to be involved in the secretory dysfunction found in Sjogren's syndrome patients and can be advantageously used as a marker that can reflect the activity of the disease.

TABLE 2

| Presence of | Number of | Mean Salivary flow | | |
|---|---|---|---|---|
| auto-antibody | patients | (95% CI) (ml/10 min) | | P |
| Anti-AQP5 IgG | | Resting salivary flow | | |
| − | 28 | 0.968 | (0.444, 1.489) | 0.003^{a} |
| + | 63 | 0.498 | (0.305, 0.692) | 0.147^{b} |
| | | Stimulated salivary flow | | |
| − | 27 | 11.085 | (7.238, 14.932) | 0.697^{a} |
| + | 61 | 10.220 | (7.787, 12.653) | 0.717^{b} |
| Anti-AQP5 IgA | | Resting salivary flow | | |
| − | 79 | 0.700 | (0468, 0.932) | 0.048^{a} |
| + | 12 | 0.267 | (−0.110, 0.643) | 0.010^{b} |
| | | Stimulated salivary flow | | |
| − | 76 | 11.111 | (8.878, 13.343) | 0.057^{a} |
| + | 12 | 6.525 | (2.200, 10.850) | 0.089^{b} |
| Anti-Ro IgG | | Resting salivary flow | | |
| − | 9 | 0.400 | (0.018, 1.489) | 0.445^{a} |
| + | 82 | 0.670 | (0.441, 0.898) | 0.512^{b} |
| | | Stimulated salivary flow | | |
| − | 8 | 9.638 | (4.753, 14.522) | 0.793^{a} |
| + | 80 | 10.570 | (8.381, 12.759) | 0.800^{b} |
| Anti-La IgG | | Resting salivary flow | | |
| − | 42 | 0.664 | (0.421, 0.908) | 0.851^{a} |
| + | 49 | 0.624 | (0.291, 0.958) | 0.288^{b} |
| | | Stimulated salivary flow | | |
| − | 40 | 10.998 | (8.148, 13.847) | 0.648^{a} |
| + | 48 | 10.058 | (7.133, 12.984) | 0.508^{b} |

In Table 2, a represents ANOVA results, b results Mann-Whitney U test results, CI represents confidence interval, and p values smaller than 0.05 were considered significant.

Although exemplary embodiments of the present disclosure have been described in detail above, the scope of the present disclosure is not limited to these embodiments, and those skilled in the art will appreciate that various modifications and improvements based on the fundamental concept of the present disclosure as defined in the appended claims also fall within the scope of the present disclosure.

Unless otherwise defined, all the technical terms used in the present disclosure have the same meaning as commonly understood by one of ordinary skill in the technical field to which the present disclosure pertains. The contents of all publications described as references herein are incorporated herein by reference.

What is claimed is:

1. A method of detecting an auto-antibody against aquaporin 5 in a biological sample of a subject in need thereof, the method comprising the steps of:
providing an aquaporin 5 antigen and the sample,
contacting the sample with the antigen; and
detecting whether the auto-antibody is present in the sample by detecting the binding between the antigen and the auto-antibody.

2. The method of claim 1, wherein the sample is a whole blood, a serum, a plasma, a tear, or a saliva.

3. The method of claim 1, wherein the antigen is provided as a transmembrane protein expressed in a cell which does not express an endogenous aquaporin 5 protein.

4. The method of claim 3, wherein the cell is a MDCK cell line.

5. The method of claim 1, wherein the antigen is provided as a soluble form comprising at least one extracellular loops of aquaporin 5.

6. The method of claim 1, wherein the antigen-antibody complex is detected by FACS (fluorescence-activated cell sorting), cell immunostaining, radial immunodiffusion, an immunoprecipitation assay including immunoelectrophoresis or countercurrent electrophoresis, RIA (radioimmunoassay) or ELISA (enzyme linked immunosorbent assay).

7. The method of claim 6, wherein the antigen-antibody complex is detected by FACS, cellular immunostaining, dipstick assay, ELISA, or lateral flow assay.

8. The method of claim 1, wherein the antigen-antibody complex is detected by use of a detection antibody, wherein the detection antibody binds specifically to a human IgG or IgA, and the detection antibody is labeled with a chromophore; an enzyme; a radioisotope; or a material comprising colored particles.

9. The method of claim 1, wherein the detection of the presence of the binding between the antigen and the auto-antibody indicates that the sample has a Sjogren's syndrome.

10. A kit for detecting or diagnosing Sjogren's syndrome, comprising an aquaporin 5 antigen and a detection antibody, wherein the detection antibody binds specifically to human IgG or IgA, and is labeled with a chromophore; an enzyme; a radioisotope; or a material comprising colored particles.

11. The kit of claim 10, wherein the aquaporin 5 antigen is bound to a solid support.

12. The kit of claim 11, wherein the solid support is a microplate, a microarray, a chip, glass, a beads or particle, or a membrane.

13. The kit of claim 10, wherein the antigen is provided as a transmembrane protein expressed in a cell which does not express endogenous aquaporin 5.

14. A composition for detecting Sjogren's syndrome, comprising an aquaporin 5 protein or its fragment or a cell that expresses the protein.

15. A method of diagnosing Sjogren's syndrome (SS) in a subject in need thereof, the method comprising:
obtaining a biological sample from the subject;
contacting the sample with an aquaporin 5 antigen, wherein the aquaporin 5 antigen is provided as a transmembrane protein expressed in a cell which does not express an endogenous aquaporin 5 protein; or the aquaporin 5 antigen is provided as a soluble form comprising at least one extracellular loops of aquaporin 5;
detecting whether the auto-antibody is present in the sample by detecting the binding between the antigen and the auto-antibody; and
diagnosing the subject having SS when the presence of the binding between the antigen and the auto-antibody is detected.

16. The method of claim 15, wherein the sample is whole blood, serum, plasma, tear, or saliva.

17. The method of claim 8, wherein the enzyme is alkaline phosphatase, biotin, beta-galactosidase, or peroxidase.

18. The method of claim 8, wherein the colored particles are colloidal gold particles or colored latex particles.

19. The method of claim 10, wherein the enzyme is alkaline phosphatase, biotin, beta-galactosidase, or peroxidase.

20. The method of claim 10, wherein the colored particles are colloidal gold particles or colored latex particles.
